(19) United States
(12) Patent Application Publication
(10) Pub. No.: US 2018/0064529 A1
(43) Pub. Date: Mar. 8, 2018

(54) IMPROVED ENDOPROSTHESIS AND CATHETER

(71) Applicant: Maxime SIBE, Le Pyla sur Mer (FR)

(72) Inventor: Maxime SIBE, Le Pyla Sur Mer (FR)

(21) Appl. No.: 15/555,410

(22) PCT Filed: Mar. 7, 2016
(86) PCT No.: PCT/EP2016/054811

§ 371 Date: Sep. 1, 2017

(30) Foreign Application Priority Data
Mar. 5, 2015 (FR) 1551883

(51) Int. Cl. A61F 2/07; A61F 2/954; A61F 2/856

(52) U.S. Cl. CPC A61F 2/07; A61F 2002/061; A61F 2/856; A61F 2/954

(57) ABSTRACT

A stent is provided comprising a tubular body formed by a polymer layer of expanded polytetrafluoroethylene and a layer made of alloy and comprising two open ends, at least one anterior side wall opening acting as an aperture allowing puncture by a needle, the side wall opening being at a predetermined distance from each of the ends and being formed in the alloy layer to allow a needle to penetrate inside the tubular body. The exact position of the anterior side wall opening within a body cavity can be identified by radiopaque markers, and the inherent nature of the expanded polytetrafluoroethylene allows puncture by a needle when it is desired to unplug a blocked or partially-blocked stent.

### BACKGROUND OF THE INVENTION

The present invention relates to an improved endoprothesis and catheter.

More particularly, the invention relates to an endoprothesis more commonly called a stent, having a mesh structure and a tubular shape adapted to be slid by means of a catheter into a human (or animal) natural cavity in order to widen it and/or keep it open.

In the state of art, when an in vivo endoprothesis or stent is partially blocked (stenosed) or even totally blocked (thrombosis), it loses its effectiveness which represents a clear danger for the person in whom the stent is provided. A surgical operation is then necessary to replace or unclog the stent obstructed by various agglomerates. The unclogging of a stent is done either by direct approach or by percutaneous endovascular means using, in particular, an expansion balloon disposed therein.

In the prior art, the teaching of document EP2077803 discloses a stent provided with a lateral opening intended to be positioned opposite a bifurcation of a blood vessel.

However, this lateral opening is neither adapted to receive, without suffering degradation, an instrument of a surgical type, nor can it be located by radiography.

This is particularly unfortunate since it complicates or even prevents subsequent cleaning out of the interior of an obstructed stent by puncturing the agglomerates with a needle. Such a de-obstruction would avoid the need of having to change the stent whenever the flow of blood within it is hindered.

Furthermore, such a stent allows the flow of fluid through its opening, which can be particularly dangerous if it leaves the location for which it was intended.

There is therefore a real need to find a solution to the technical problem previously set forth.

### SUMMARY OF THE INVENTION

To this end, the present invention provides a self-expanding stent comprising a tubular body, said tubular body being formed by at least one polymer layer of circular cross-section and a layer made of alloy of circular cross-section and comprising two open ends, at least one anterior side wall opening acting as an aperture allowing puncture by a needle and having a contour, said anterior side wall opening being arranged at a predetermined distance from each of said ends and being formed in the alloy layer to allow a needle to penetrate into said tubular body, and at least one closure member for said side wall opening provided in the polymer layer, said closure being at least partial.

By virtue of these arrangements, after placement of the stent, a surgeon can introduce a surgical instrument through the anterior side wall opening and thus perform at least an agglomerate puncture and/or a cleaning of the internal wall of the stent.

The self-expansibility of the stent according to the invention enables it to adapt to different arterial diameters. In particular, the stent according to the invention allows endovascular treatment of a contralateral common femoral artery or, via said common femoral artery, another artery such as a coronary artery, a digestive artery, the aorta or a sub-inguinal artery, whether by retrograde or anterograde route.

According to one embodiment of this stent, the closure member includes an orifice adapted to allow a needle to penetrate into the tubular body.

In a preferred embodiment,
the polymer layer is internal and the alloy layer is external,
the polymer is expanded polytetrafluoroethylene and the alloy is Nitinol.

The term Nitinol should be understood to mean an alloy of nickel and titanium, in which these two elements are present in approximately equal percentages.

The inventor has determined that these arrangements are optimal.

According to yet another embodiment of this stent, at least one radiopaque marker is arranged at the periphery of said anterior side wall opening.

By virtue of this arrangement, the location of the anterior side wall opening can be identified by radiography and the surgeon can minimize the size of incision necessary to reach it.

According to one particular embodiment, the side wall openings are arranged in staggered fashion along the tubular body.

By virtue of this arrangement, despite the multiplicity of side wall openings, the stent has sufficient mechanical strength to keep the natural cavity within which it is installed open.

According to yet another embodiment of this stent:
one end of said tubular body is bifurcated to form two tubular branches each having an open end,
the polymer layer of the tubular body and the tubular branches is of a one-piece construction,
the alloy layer of the tubular body and of the tubular branches is of a one-piece construction.

This particular one-piece structure of the stent allows it to be placed at a bifurcation in a natural cavity.

According to a preferred feature, each tubular branch includes at least one radiopaque marker at its open end. This allows, where the stent is provided at a bifurcation in a natural cavity, the position and the orientation of its branches to be determined by radiography.

According to preferred features,
the cross-section of the tubular branches and of the tubular body is circular,
the cross-section of the tubular body has a diameter of between 7 mm and 11 mm and a length of the tubular body is between 30 and 40 mm,
the cross-section of one of the two tubular branches, referred to as the shorter branch, has a diameter of between 3 mm and 5 mm, and the length of the shorter branch is between 10 mm and 15 mm, and
the cross-section of the other of the two tubular branches, referred to as the longer branch, has a diameter of between 5 mm and 6 mm, and a length of the longer branch is of order of 20 mm.

The inventor has determined that these arrangements are optimal.

The invention also provides an assembly comprising a stent and a catheter, the catheter including a tubiform body, in which:
the tubiform body includes at least four channels extending therein and two ends each extended by two tubular branches, the ends of which are open,
each tubular branch of a first end of the catheter includes a bifurcation terminating in a stent delivery device,
two of the channels each carry a wire forming a guide wire emerging from one of the open ends,
the other two channels each carry a stent delivery wire, a first end of which is connected to the delivery device.
the second end of the delivery wires being connected to the stent.

The advantages associated with the catheter for delivering the stent being similar to those of the stent, these are not repeated here.

The invention also provides an assembly comprising such a stent and such a catheter, in which a second end of the delivery wires is fastened to the stent.

Finally, the invention relates to the use of such an assembly.

Other characteristics and advantages of the invention will appear on reading the following description of a preferred embodiment of the invention, given by way of example and with reference to the accompanying drawings.

### BRIEF DESCRIPTION OF THE DRAWINGS

FIG. 1 is an oblique perspective view of a first example of a unibody stent according to the invention;

FIG. 2 is an oblique perspective view of a second example of a unibody stent according to the invention;

FIG. 3 shows an overall view in profile of an example of an assembly comprising a unibody stent and an improved catheter according to the invention.

### DETAILED DESCRIPTION OF PREFERRED EMBODIMENTS

In the description which follows, identical element or elements having an identical function bear the same reference sign. For the sake of concision of the present description, elements which are identical in the various examples are not described again with regard to each of these examples. In other words, only differences between the various examples are described in detail, common elements being described with reference to a single example.

Furthermore, numerical values given hereunder, although not limiting, have been found as a result of testing to provide the most advantageous results.

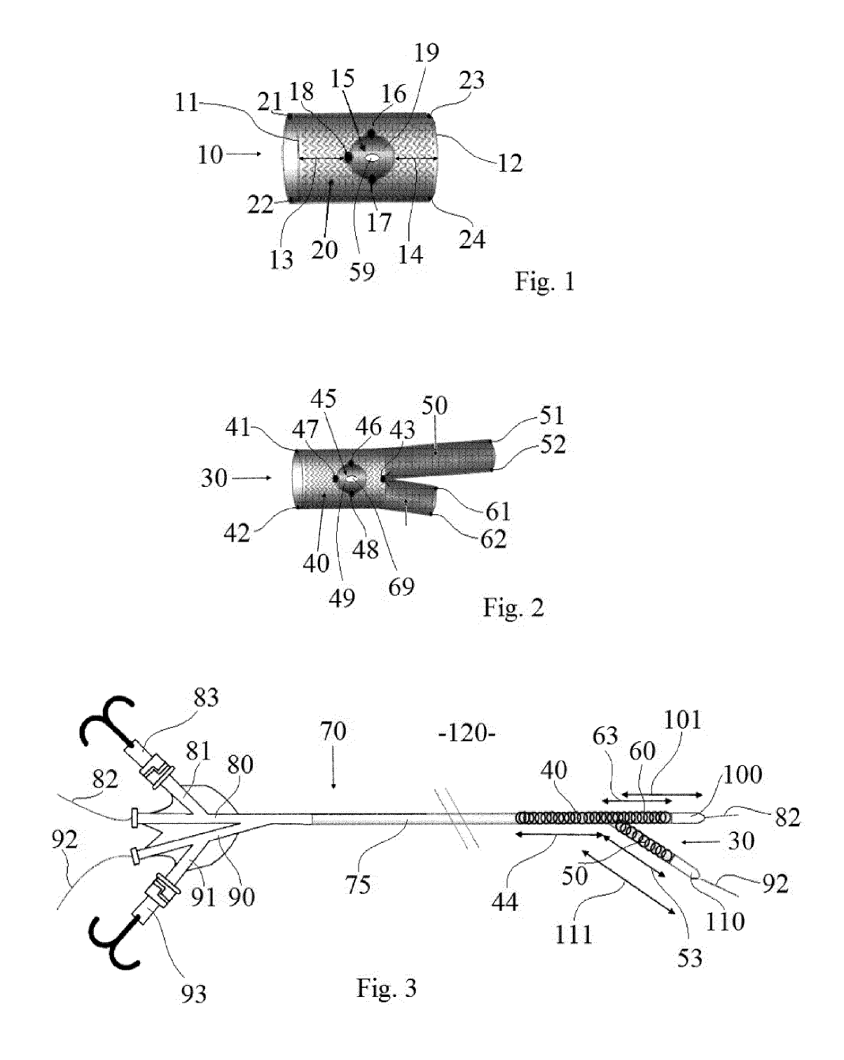

FIG. 1 shows a first embodiment of a self-expanding stent according to the invention, referred to as stent 10.

The stent 10 comprises a tubular body 20 which has two open ends 11 and 12.

In the example, the cross-section of the tube formed by the body 20 is circular. In alternative embodiments, this cross-section is of another form, for example it may be of any desired shape. Preferably, the cross-section of tubular body 20 has a diameter of between seven millimeters and eleven millimeters and its length is between thirty and forty millimeters.

In the example, stent 10 comprises a polymer layer and a layer made of alloy.

Preferably, the polymer layer is internal and the alloy layer is external. In an alternative embodiment, the polymer layer is external and the alloy layer is internal. Typically, the polymer is expanded polytetrafluoroethylene (ePTFE) and the alloy is Nitinol.

In the example, an anterior side wall opening 15 which is referred to herein as the needle puncture aperture is provided on the stent 10 and is arranged towards the middle of tubular body 20.

In alternative embodiments, the stent according to the invention has several side wall openings in which case these are arranged staggered along the tubular body so as to retain sufficient mechanical strength for the stent to keep the natural cavity within which the stent is installed open.

In the example, the stent 10 includes an element 19 for partially obturating needle puncture aperture 15. This obturation is partial since closure member 19 has an orifice 59 adapted to allow an instrument or tool of the surgical type to penetrate inside tubular body 20.

In an alternative preferred embodiment, there is no such orifice provided in the closure member and in place of this the expanded polytetrafluoroethylene (ePTFE) is present at this region while the external Nitinol layer is absent at this region. The elasticity and strength of the expanded polytetrafluoroethylene enables it to be pierced by a needle a number of times without it getting torn or otherwise deteriorated.

Typically, anterior side wall opening 15 is provided in the alloy layer at a predefined distance 13, 14 from each of the respective ends 11, 12 of tubular body 20. Preferably each distance 13, 14 is greater than a maximum dimension of anterior side wall opening 15. Thus, the mechanical strength discussed above is assured. In the example, the contour of anterior side wall opening 15 being substantially circular, the maximum dimension of said opening is its diameter.

Typically, closure member 19 is formed in the polymer layer which has an elasticity favoring the introduction of a surgical instrument through orifice 59.

In the example, three radiopaque markers 16, 17 and 18 are arranged at the periphery of anterior side wall opening 15. In this way, the contour of opening 15 can be located by radiography when the stent 10 is disposed in a human natural cavity. Advantageously, the use of at least one brightness enhancer makes it possible to increase the precision of radioscopic images representing the stent according to the invention.

In the example, two radiopaque markers 21-22 and 23-24 are provided at each open end 11, 12 respectively of tubular body 20. Thus, the space occupied by stent 10 can be visualized by radiography when it is disposed in a human natural cavity.

In alternative embodiments, the number and nature of the markers are different, but they have the same function.

FIG. 2 shows a second embodiment of a self-expanding stent according to the invention, referred to as stent 30.

The stent 30 comprises a tubular body 40 with two open ends. In the example, the cross-section of the tube formed by tubular body 40 is circular. In alternative embodiments, this cross-section is of another form, for example, it may be of any appropriate shape. Preferably, the cross-section of tubular body 40 has a diameter of between seven millimeters and eleven millimeters and its length 44, shown in FIG. 3, is between thirty and forty millimeters.

In the example, stent 30 comprises a polymer layer and a layer made of alloy. Preferably, the polymer layer is internal and the alloy layer is external. In an alternative embodiment, the polymer layer is external and the alloy layer is internal.

Typically, the polymer is expanded polytetrafluoroethylene (ePTFE) and the alloy is Nitinol.

In the example, an anterior side wall opening 45 providing an aperture for needle puncture is provided on the stent 30, towards the middle of tubular body 40.

In alternative embodiments, the stent according to the invention has several side wall openings, in which case these are arranged staggered along the tubular body so as to retain sufficient mechanical strength for the stent to keep the natural cavity within which it is installed open.

In the example, stent 30 includes an element 49 for partially obturating opening 45. This obturation is, here, partial because the closure member 49 has an orifice 69 adapted to allow a surgical instrument to penetrate into the opening of tubular body 40.

Typically, the anterior side wall opening is formed in the alloy layer at a predefined distance from each of the ends of tubular body 40, preferably a distance greater than a maximum dimension of side wall opening 45. In this way, the mechanical strength previously described is maintained. In the example, the contour of side wall opening 45 being substantially circular, the maximum dimension of said opening is its diameter.

The closure member 49 is formed in the polymer layer which has an elasticity which is favorable to the introduction of a surgical instrument through the orifice 69.

In the example, three radiopaque markers 46, 47 and 48 are provided at the periphery of anterior side wall opening 45. Thus, the contour of opening 45 can be located by radiography when the stent 30 is disposed in a human natural cavity.

In the example, two radiopaque markers 41 and 42 are provided at one of the two open ends of tubular body 30. The other end of tubular body 40 opens out into two tubular branches 50 and 60 each having an open end.

A marker 43 is disposed at the end of tubular body 40 where it diverges to form the two tubular branches 50 and 60, between the said branches.

Each tubular branch 50, 60 includes at least one radiopaque marker 51, 52, 61, 62 at its open end.

Thus, the area occupied by the stent 30 can be precisely determined by radiography when it is disposed in a human natural cavity.

In alternative embodiments, the number and nature of the markers are different, but they have the same function.

Typically, each of the said tubular branches 50, 60 comprises at least one polymer layer and one alloy layer. In other words, the polymer layer of tubular body 40 and of the tubular branches 50 and 60 of stent 30 has a one-piece construction.

Likewise, the alloy layer of tubular body 40 and the tubular branches 50 and 60 of stent 30 has a one-piece construction.

Typically, the cross-section of the branches 50 and 60 is circular.

Preferably, the cross-section of one of the two tubular branches 50 and 60, referred to herein as the shorter branch 60, has a diameter of between three and five millimeters and the length 63 shown in FIG. 3 of said shorter branch is between ten and fifteen millimeters.

The cross-section of the other one of the two tubular branches 50 and 60, referred to herein as the longer branch 50, has a diameter of between five and six millimeters and the length 53 shown in FIG. 3 of said longer branch is of order of twenty millimeters.

FIG. 3 shows an embodiment of an assembly 120 comprising stent 30 and a catheter 70 for delivering the stent, according to the invention.

Catheter 70 has a tubiform body 75. Tubiform body 75 includes at least four channels (which are not illustrated) extending therein, and two ends, each of which is prolonged by two tubular branches 80, 90, and respectively 100 and 110, the ends of which are open.

Each tubular branch 80, 90 at a first end of catheter 70 has a bifurcation 81, 91 terminated by a stent delivery device 83, 93.

The two branches 100 and 110 of a second end of catheter 70 are designed to have the tubular branches 60 and 50 respectively of stent 30 wrapped around them and have a length 101, 111 of between 2.5 cm and 3.5 cm.

Two of the channels of catheter 70 extend over the entire length of tubular branches 80 and 90 of tubiform body 75 as well as of the tubular branches 100 and 110 so as to each be able to receive a guide wire 82, 92, which exits through the open ends of the said four tubular branches.

The other two channels of catheter 70 each carry a so-called delivery wire (not shown), one end of which is connected to the delivery device 83, 93 with a second end of the delivery wires being connected to the stent 30.

Preferably the delivery wires form a trussed structure around stent 30 and are arranged so that when said wires are pulled by means of the delivery devices 83 and 93:
a distal portion of the end of said wires is the first to detach from said stent, thereby beginning to untruss said stent, and
a proximal portion of the end of said wires then detaches from said stent, which ends up with the release of said stent.

The term “distal portion” means herein that portion of the end of the delivery wires which is furthest from the delivery devices 83 and 93. Correspondingly, the term “proximal portion” as used herein means that portion of the end of the wires which is closest to the delivery devices 83 and 93.

The invention also relates to the use of such an assembly 120.

Obviously, the present invention is not limited to the examples described and shown, but may be is the subject of numerous alternative embodiments accessible to those skilled in the art.

1. A self-expanding stent comprising a tubular body, said tubular body being formed by at least one polymer layer of circular cross-section and a layer made of alloy of circular cross-section and comprising:
two open ends,
at least one anterior side wall opening acting as an aperture allowing puncture by a needle and having a contour, said anterior side wall opening being arranged at a predetermined distance from each of said ends and being formed in the alloy layer to allow a needle to penetrate into said tubular body, and
at least one closure member for said side wall opening provided in the polymer layer, said closure being at least partial.

2. The stent according to claim 1, wherein the closure member includes an orifice adapted to allow a needle to penetrate into the tubular body.

3. The stent according to claim 1, wherein:
the polymer layer is internal and the alloy layer is external,
the polymer is expanded polytetrafluoroethylene and the alloy is Nitinol.

4. The stent according to claim 1, wherein at least one radiopaque marker is arranged at the periphery of said side wall opening.

5. The stent according to claim 1, wherein the side wall openings are arranged in a staggered manner along said tubular body.

6. The stent according to claim 1, wherein at least one radiopaque marker is provided at each open end of said tubular body.

7. The stent according to claim 1, wherein:
one end of said tubular body is bifurcated to form two tubular branches each having an open end,
the polymer layer of said tubular body and of said tubular branches is of a one-piece construction,
the alloy layer of said tubular body and of said tubular branches is of a one-piece construction.

8. The stent according to claim 7, wherein each tubular branch includes
at least one radiopaque marker at its open end,
at least one polymer layer and one alloy layer.

9. The stent according to claim 7 wherein:
a cross-section of said tubular branches and of said tubular body is circular,
a cross-section of said tubular body has a diameter of between 7 mm and 11 mm and a length of said tubular body is between 30 and 40 mm,
a cross-section of one of said two tubular branches, referred to as the shorter branch, has a diameter of between 3 mm and 5 mm, and a length of said shorter branch is between 10 mm and 15 mm;
a cross-section of the other of said two tubular branches, referred to as the longer branch, has a diameter of between 5 mm and 6 mm, and a length of said longer branch is of order of 20 mm.

10. The stent according to claim 1, wherein a contour of said anterior side wall opening is substantially circular, a maximum dimension of said opening being its diameter.

11. An assembly comprising a stent according claim 7 and a catheter, said catheter including a tubiform body, wherein
said tubiform body includes at least four channels extending therein and two ends each extended by two tubular branches, the ends of which are open,
each tubular branch of a first end of said catheter includes a bifurcation terminating in a stent delivery device
each tubular branch of a second end of said catheter is adapted to be enveloped by one of said tubular branches of said stent
two of said channels each carry a wire forming a guide wire emerging from one of said open ends,
the other two channels each carry a delivery wire, a first end of which is connected to the delivery device, a second end of the delivery wires being connected to the stent.
